(19) United States
(12) Patent Application Publication
(10) Pub. No.: US 2006/0070628 A1
(43) Pub. Date: Apr. 6, 2006

(54) Tracheostomy apparatus

(75) Inventors: Joanna Byatt, Hythe (GB); Adam Tebbutt, Hythe (GB); Barnaby Townsend, Oxford (GB)

Correspondence Address: LOUIS WOO;LAW OFFICE OF LOUIS WOO, 717 NORTH FAYETTE STREET, ALEXANDRIA, VA, 22314, US

(73) Assignee: Smiths Group plc, London (GB)

(21) Appl. No.: 11/229,647

(22) Filed: Sep. 20, 2005

(30) Foreign Application Priority Data
Oct. 2, 2004 (GB) 0421938.2

(51) Int. Cl. A62B 9/06; A61M 16/00

(52) U.S. Cl. 128/207.29; 128/207.14; 128/207.15

(57) ABSTRACT

Tracheostomy apparatus includes a needle with a doppler ultrasound transducer mounted within it close to its patient end and connected to a monitor. Two electrodes are insulated from one another on the external surface of the needle close to its patient end. The electrodes are also connected to the monitor. The needle supports a catheter on its outer surface. As the needle is inserted through tissue overlying the trachea, the ultrasound transducer detects the presence of any nearby blood vessels and gives an audible indication on the monitor. The monitor also responds to a rise in impedance between the electrodes indicative of entry of the end of the needle into the trachea so that the needle can be removed to leave the catheter in place. A guide is then inserted through the catheter and the path into the trachea is enlarged with a dilator sufficiently to receive a tracheostomy tube.

### BACKGROUND OF THE INVENTION

This invention relates to tracheostomy apparatus.

Many tracheostomies are performed routinely, to aid breathing or ventilation of patients, without problem. However, difficulties can arise in some cases. Whereas in most patients the major blood vessels are located away from the region where the tracheostomy is performed, some patients do have major blood vessels in this region. If there is no way to identify the location of such blood vessels they may inadvertently be cut during the tracheostomy procedure. This is a particular problem because of the difficulty of stemming blood flow from a cut blood vessel in this region. The loss of blood can result in death. Another problem arises, particularly in the percutaneous tracheostomy procedure, of ensuring that the trachea is correctly located by the introducing instruments so that the patient end of the tracheal tube locates within the trachea and not between layers of tissue overlying the trachea. One solution to this problem is provided by the arrangement described in GB2393398.

### BRIEF SUMMARY OF THE INVENTION

According to one aspect of the present invention there is provided tracheostomy apparatus including electrical means arranged to provide feedback to the user of the presence of a feature in the region of the tracheostomy.

The feature may be the trachea and or alternatively blood vessels. The electrical means may include an acoustic device arranged to propagate acoustic energy into tissue overlying the trachea and to receive reflected energy. The acoustic device preferably includes an ultrasound transducer and is preferably arranged to detect doppler shift in the frequency of reflected energy. The apparatus may include a needle with a puncturing tip, the acoustic device being located within the needle. The apparatus may include a catheter supported on and extending along the needle so that the needle can be withdrawn after entry to the trachea to leave the catheter in place. The electrical means may include an electrode close to the patient end of the apparatus arranged to contact tissue during penetration of tissue overlying the trachea and to be exposed when the patient end is located in the trachea. The electrode is preferably exposed on an external surface of the needle. The apparatus may include two electrodes insulated from one another and both arranged to contact tissue during penetration of tissue, the electrical means being arranged to monitor the impedance between the two electrodes.

According to another aspect of the present invention there is provided tracheostomy apparatus including a probe, an acoustic transducer located with the probe and arranged to provide feedback to the user indicative of the presence of blood vessels during insertion of the probe through tissue.

According to a further aspect of the present invention there is provided tracheostomy apparatus including a probe adapted to penetrate tissue overlying the trachea until the patient end of the probe penetrates the trachea, the probe including electrode means close to its patient end adapted to contact tissue overlying the trachea during passage through the tissue and to be exposed when the patient end is located within the trachea, and electrical means for monitoring the change in impedance as the electrode means moves from contact with the tissue to being exposed within the trachea and for providing feedback to the user when the patient end of the probe enters the trachea.

The probe may include both an acoustic transducer and electrode means, the apparatus including a monitor arranged to indicate both penetration of the trachea and the presence of blood vessels. The apparatus may include a catheter extending along the external surface of the probe so that the probe can be withdrawn after entry into the trachea to leave the catheter in place, an elongate guide member insertable within the catheter after removal of the probe, a dilator or dilators slidable along the guide member to enlarge the opening into the trachea, and a tracheostomy tube slidable along the guide so that its patient end locates in the trachea.

According to a fourth aspect of the present invention there is provided tracheostomy apparatus including first electrical means for indicating entry into the trachea and second electrical means for indicating proximity of blood vessels.

The feedback to the user is preferably in audible form. The apparatus may include an acoustic probe arranged to be moved over the external surface of the tissue overlying the trachea prior to making an incision.

Tracheostomy apparatus according to the present invention will now be described, by way of example, with reference to the accompanying drawings.

### BRIEF DESCRIPTION OF THE DRAWINGS

FIG. 1 is a cross-sectional view of the needle assembly during insertion through neck tissue;

FIG. 2 is a cross-sectional view of the patient end tip of the assembly to a larger scale;

FIG. 3 is a cross-sectional view of the apparatus at a preliminary stage;

FIG. 4 is a cross-sectional view of the apparatus with the patient end of the needle assembly located in the trachea; and

FIGS. 5 to 10 are cross-sectional view of subsequent steps in use of the apparatus.

### DETAILED DESCRIPTION OF THE PREFERRED EMBODIMENT

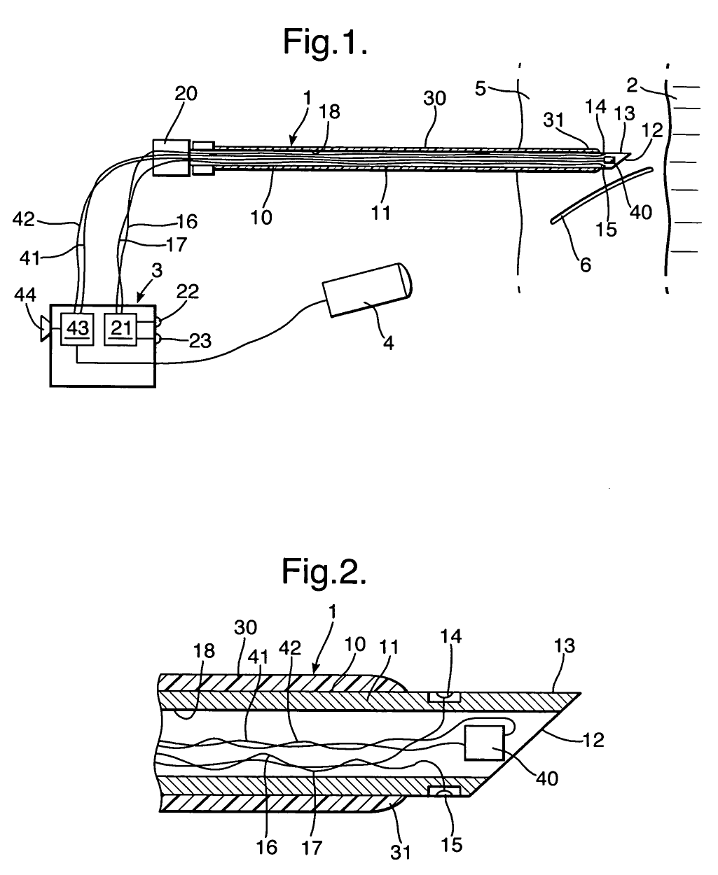

With reference first to FIGS. 1 and 2, the apparatus includes a probe in the form of a cutting device provided by a needle assembly 1 used to make the initial entry into the trachea 2 and an electrical feedback unit 3 operable to provide feedback to the user of features in the region of the tracheostomy, in particular, of the trachea itself and of large blood vessels. The apparatus also includes an external doppler ultrasound handset 4 connected with the feedback unit 3, which is used for preliminary investigations.

The probe or needle assembly I includes an inner needle 10 having a rigid, tubular cannula or shaft 11 of a metal and with a bevelled cutting or puncturing tip 12 at its patient end 13. Although the needle is shown as being hollow, it could be solid. On the outer surface of the shaft 11 close to the patient end 13 are two electrodes 14 and 15 spaced from one another around the circumference of the shaft. The electrodes 14 and 15 are exposed on the outside of the shaft 11 and are electrically insulated from one another and from the shaft 11. The electrodes 14 and 15 are electrically connected with respective wires 16 and 17, which extend through the wall of the shaft 11 and pass rearwardly along the bore 18 of the needle 10. The wires 16 and 17 emerge from a plastics hub 20 at the machine end of the needle 10 and extend to an impedance-measuring circuit 21 within the feedback unit 3. The impedance circuit 21 acts to monitor the impedance between the two electrodes 14 and 15 using any conventional dc or ac impedance measuring arrangement. While the electrodes 14 and 15 are both in contact with the tissue 5 overlying the trachea 2, the impedance between the electrodes is relatively small because of the relatively high conductivity of the tissue. When the electrodes 14 and 15 emerge into the trachea 2, the impedance rises. The impedance-measuring circuit 21 is arranged to provide a warning alarm signal, such as an audible signal on a buzzer 22 or the like, or a visible signal on a light 23 or the like, or both.

The needle assembly 1 also includes a plastics catheter 30 extending along the outside of the needle shaft 11 as a close fit. The patient end 31 of the catheter 30 terminates close to the patient end 13 of the needle 10 and just to the rear of the electrodes 14 and 15. The patient end 31 of the catheter 30 is tapered to form a smooth transition with the outside of the needle shaft 11 so as to ease passage through the neck tissue 5.

The needle assembly 1 is completed by an ultrasound acoustic transducer 40 mounted within the bore 18 of the needle 10 close to its patient end 13. The transducer 40 may be of any conventional kind suitable for use with doppler ultrasound equipment and would typically include a piezoelectric element. The transducer 40 is oriented so that it directs acoustic energy axially forwardly out of the tip 13 of the needle 10 and so that it is responsive to reflected energy directed back along the same axis. The transducer 40 is connected with wires 41 and 42 by which electrical energy is supplied to and from the transducer. The wires 41 and 42 extend rearwardly along the bore 18 of the needle 10 and emerge out of its hub 20 where they extend to a doppler ultrasound circuit 43 in the feedback unit 3. The doppler ultrasound circuit 43 provides pulsed electrical energy to the transducer 40 to cause it to propagate ultrasonic energy, in the usual way. The ultrasound circuit 43 is responsive to the variations in frequency of the received energy caused by reflection from moving reflectors and, in particular, from blood flowing along blood vessels. After suitable processing, the received energy is converted into an audible signal that is supplied to a loudspeaker 44 on the unit 3. The loudspeaker 44 provides a sound representative of blood flow along a blood vessel if the needle tip 13 is near and is directed at a blood vessel.

Different frequencies can be used for the doppler ultrasound function and the impedance function so it may be possible to use common wires for the electrodes 14 and 15 and the transducer 40 by providing suitable filters to select between the different signals.

It is preferable for the feedback provided by the unit 3 to the user to be of a kind that can be perceived without the user having to look away from the surgical site, such as an audible feedback or a flashing light that can be seen by peripheral vision.

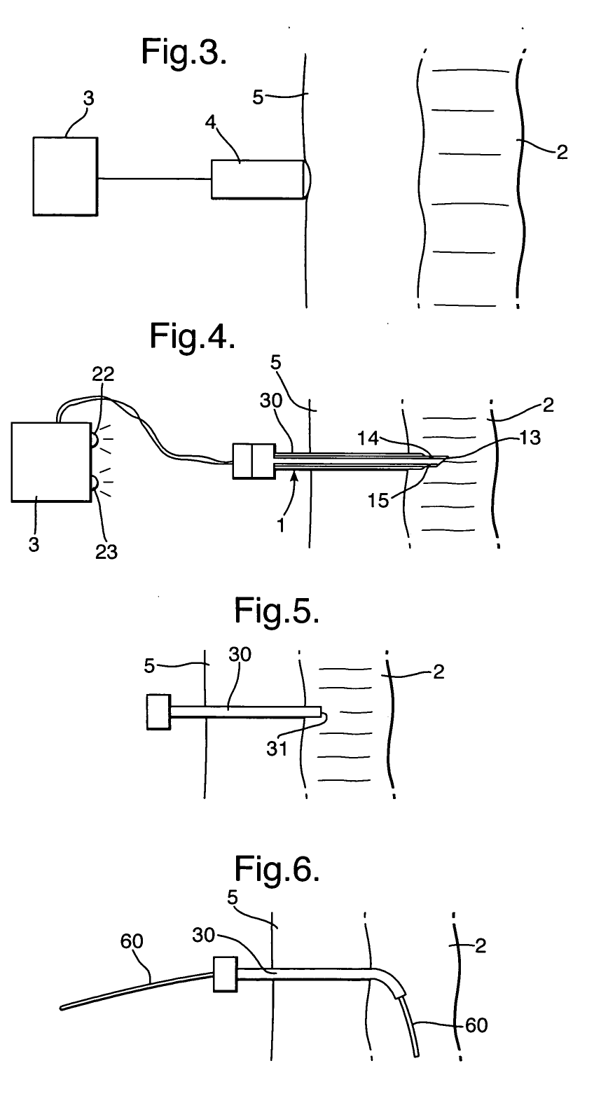

The procedure in which the apparatus is used will now be described in greater detail with reference also to FIGS. 3 to 10.

As shown in FIG. 3, before any incision is made, the external handset 4 is placed against the skin of the neck in the region where it is proposed to form the tracheostomy. A gel may be applied to the skin, in the usual way when carrying out ultrasound observation, to improve the acoustic transmission between the probe 4 and the underlying tissue. The probe 4 is angled along the proposed path of the tracheostomy incision into the trachea 2. The user listens to the feedback unit 3 to determine if there are any sounds indicative of pulsing blood flow. If such sounds are heard, the user repositions the probe 4 and tries again until he finds a suitable location that appears free of blood vessels. The user also listens for the characteristic sound made by air moving in and out along the trachea so that he can determine the position of the trachea. When a suitable location and path into the trachea has been identified, the user then proceeds to insert the needle assembly 1.

FIG. 1 shows the needle assembly 1 being advanced forwardly through neck tissue 5 overlying the trachea 2. As long as the tip 13 of the assembly 1 remains in contact with the tissue 5 the impedance circuit 21 does not produce any warning output. The doppler ultrasound circuit 43 may be arranged to provide a continuous audible output on the speaker 44. In normal use, the sound produced will be at a low level and will characteristic of movement through the tissue 5. If, however, the tip 13 should come adjacent and in alignment with a large blood vessel 6, the sound would change to a characteristic pulsating, susurrating sound similar to that of waves on a shore, indicative of arterial blood flow, or a more constant whooshing sound indicative of venous flow. If the user hears this sound he knows immediately to withdraw or redirect the needle assembly 1. The ultrasound circuit 43 could be arranged to mute all sound from the loudspeaker 44 until a blood vessel 6 were detected. Instead of producing a direct audible representation of the sound of the blood flow, the ultrasound circuit could include recognition software to identify the characteristic signal and then provide a warning alarm such as by a buzzer or flashing light. The needle assembly could include a mute button to enable the user manually to turn the ultrasound on or off.

If the user hears no warning sound from the ultrasound circuit 43 he continues advancing the needle assembly 1 until he hears the warning alarm from the impedance circuit 21 to indicate that the trachea 2 has been entered, as shown in FIG. 4. The audible output of the ultrasound circuit 43 will also change when the trachea 2 is penetrated, because of the reduced ultrasound transmissivity of air compared with tissue. This will be apparent to the user because the sound from the loudspeaker 44 will become quieter when the tip 13 of the assembly 1 enters the trachea 2

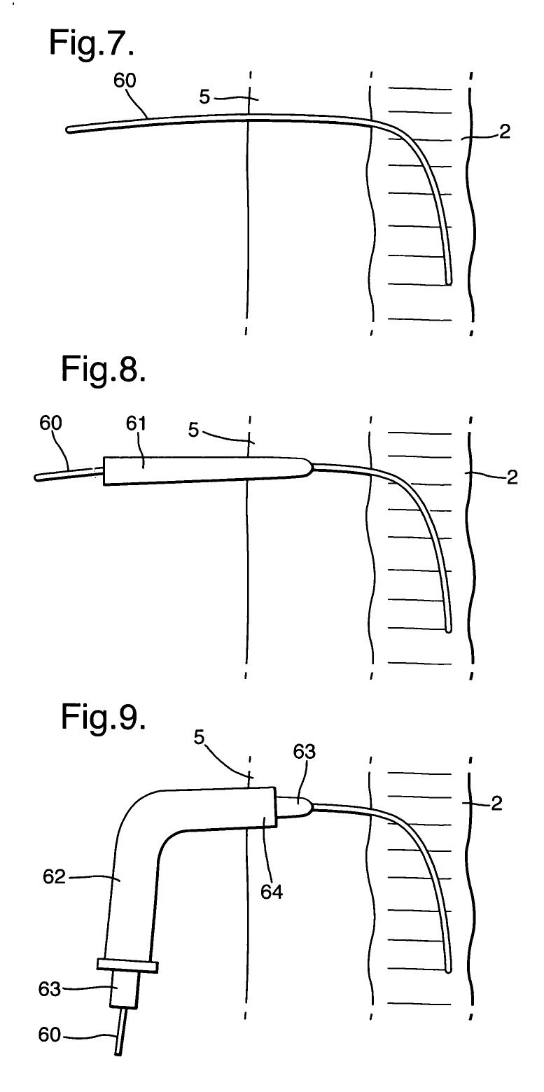

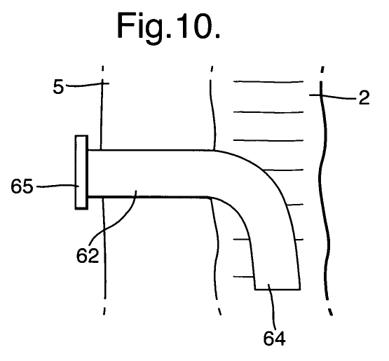

As shown in FIG. 5, the user then removes the needle 10 but leaves the catheter 30 in place. Next, as shown in FIG. 6, he inserts a guidewire 60, or other elongate guide member, through the catheter 30 and, once this has been done, the catheter can be removed, as shown in FIG. 7, leaving the guidewire in place. FIG. 8 shows the next step, which is to push a dilator 61 along the guidewire 60 to enlarge the diameter of the passage through the neck tissue 5. A single dilator, such as described in GB2394669, or a series of dilators of increasing diameter could be used. A tracheostomy tube 62, mounted on an introducer 63, is then pushed along the guidewire 60, as shown in FIG. 9. Once fully inserted, the guidewire 60 and introducer 63 are removed, leaving the tracheostomy tube 62 with its patient end 64 located in the trachea 2 and its machine end 65 adjacent the surface of the neck, as shown in FIG. 10.

It will be appreciated that the apparatus of the present invention can enable the safety of tracheostomy procedures to be improved by reducing the risk of damage to blood vessels and reducing the risk that the tracheostomy tube will be incorrectly located. The apparatus may also enable tracheostomy procedures to be completed more quickly because it can give the user greater confidence in avoiding injury to the patient. The speed at which a tracheostomy is performed can be important where the patient has compromised breathing.

Various modifications are possible to the apparatus. For example, the impedance measuring electrodes could be mounted on the catheter instead of the needle. One electrode could be provided by the end of the needle protruding from the catheter and the other electrode could be separate and be applied, for example, to the patient's skin. Other versions of the apparatus could include the impedance measuring circuit without the ultrasound function, or could include the ultrasound circuit without the impedance function.

The probe used to form the path through the neck tissue need not be a needle or other cutting device but could be provided by a blunt dissector. With a blunt dissector, an initial cut would be made through the skin, such as with a scalpel, and the dissector would be pushed through the neck tissue up to the wall of the trachea. A retractable cutting device within the dissector would then be exposed to enable the wall of the trachea to be penetrated.

What we claim is:

1. Tracheostomy apparatus comprising a probe and an electrical device arranged to provide feedback to a user indicative of the presence of a feature in a region of a tracheostomy.

2. Apparatus according to claim 1, wherein the feature includes the trachea.

3. Apparatus according to claim 1, wherein the feature includes blood vessels.

4. Apparatus according to claim 1, wherein the electrical device includes an acoustic device arranged to propagate acoustic energy into tissue overlying the trachea and to receive reflected energy.

5. Apparatus according to claim 4, wherein the acoustic device includes an ultrasound transducer and is arranged to detect doppler shift in the frequency of reflected energy.

6. Apparatus according to claim 4 including a needle with a puncturing tip, and wherein said acoustic device is located within said needle.

7. Apparatus according to claim 6, wherein the apparatus includes a catheter supported on and extending along said needle so that said needle can be withdrawn after entry to the trachea to leave the catheter in place.

8. Tracheostomy apparatus comprising a probe, an acoustic transducer located with the probe and a monitor arranged to provide feedback to a user indicative of presence of blood vessels during insertion of said probe through tissue.

9. Apparatus according to claim 1, wherein the electrical device includes an electrode close to the patient end of the probe, and wherein said electrode is arranged to contact tissue during penetration of tissue overlying the trachea and to be exposed when a patient end of said probe is located in the trachea.

10. Apparatus according to claim 9, including two electrodes insulated from one another and both arranged to contact tissue during penetration of tissue, and wherein the electrical device is arranged to monitor impedance between the two electrodes.

11. Apparatus according to claim 9 including a needle with a puncturing tip, and wherein said electrode is exposed on an external surface of said needle.

12. Apparatus according to claim 9, wherein the apparatus includes a catheter supported on and extending along said probe so that said probe can be withdrawn after entry to the trachea to leave the catheter in place.

13. Tracheostomy apparatus comprising: a probe having a patient end adapted to penetrate a trachea through tissue overlying the trachea; an electrode mounted on said probe close to its patient end and adapted to contact tissue overlying the trachea during passage through the tissue and to be exposed when said patient end is located within the trachea; and an electrical monitor arranged to monitor change in impedance as said electrode moves from contact with the tissue to being exposed within the trachea and for providing feedback to a user when said patient end of said probe enters the trachea.

14. Tracheostomy procedure apparatus according to claim 13 including a catheter extending along the external surface of said probe so that said catheter can be left in place after removal of the probe, an elongate guide member insertable within said catheter after removal of said probe, a dilator or dilators slidable along said guide member to enlarge an opening into the trachea, and a tracheostomy tube slidable along said guide member so that a patient end of said tracheostomy tube locates in the trachea.

15. Tracheostomy apparatus comprising: a needle with a cutting, patient end tip adapted to penetrate tissue in the region of the trachea; a doppler acoustic device located within said needle towards said patient end; and a monitor connected with said doppler acoustic device and arranged to provide feedback to a user indicative of the presence of blood vessels in said tissue.

16. Apparatus according to claim 15 including a catheter extending along an external surface of said needle so that said needle can be withdrawn after entry into the trachea to leave said catheter in place; an elongate guide member insertable within said catheter after removal of said probe; a dilator or dilators slidable along said guide member to enlarge an opening into the trachea; and a tracheostomy tube slidable along said guide member so that a patient end of said tube locates in the trachea.

17. Tracheostomy apparatus including a first electrical device arranged to indicate entry into the trachea and a second electrical device arranged to for indicate proximity of blood vessels.

18. Apparatus according to claim 1, wherein said feedback to the user is in an audible form.

19. Apparatus according to claim 1 including an acoustic probe arranged to be moved over an external surface of the tissue overlying the trachea prior to making an incision.

20. A method of performing a tracheostomy including the steps of: inserting a probe including an acoustic transducer into tissue overlying the trachea, monitoring for acoustic signals indicative of blood flow through blood vessels and continuing insertion only in the absence of such signals.

21. A method according to claim 20 including a preliminary step of monitoring externally in a region of proposed insertion of said probe for acoustic signals indicative of blood flow through blood vessels.

22. A method of performing a tracheostomy including the steps of: monitoring externally with an acoustic probe for acoustic signals indicative of blood flow through blood vessels, identifying a path into the trachea that is substantially free of major blood vessels and subsequently forming a tracheostomy along said path.

23. A method according to claim 22 including the step of monitoring for signals indicative of air flow along the trachea to identify the position of the trachea.

24. A method of performing a tracheostomy including the steps of: inserting a probe including an impedance electrode into tissue overlying the trachea, and monitoring impedance at the electrode until there is a rise in impedance indicative of penetration of the trachea.

25. A method of performing a tracheostomy including the steps of: inserting a probe including an acoustic transducer and an impedance electrode into tissue overlying the trachea, monitoring for acoustic signals indicative of blood flow through blood vessels, monitoring impedance at the electrode, and continuing insertion of the probe in the absence of signals indicative of the presence of blood vessels and in the absence of a rise in impedance at the electrode indicative of penetration of the trachea.
